(19) United States
(12) Patent Application Publication
(10) Pub. No.: US 2014/0357870 A1
(43) Pub. Date: Dec. 4, 2014

(54) PROCESS FOR THE PREPARATION OF DEXLANSOPRAZOLE

(71) Applicants: Anmol Kumar Ray, Jaipur (IN); Anu Mittal, Kurukshetra (IN); Nagaraju Gottumukkala, West Godavari (IN); Mahavir Singh Khanna, New Delhi (IN); Rajesh Kumar Thaper, Jammu (IN); Mohan Prasad, Gurgaon (IN); Sudershan Kumar Arora, Gurgaon (IN)

(72) Inventors: Anmol Kumar Ray, Jaipur (IN); Anu Mittal, Kurukshetra (IN); Nagaraju Gottumukkala, West Godavari (IN); Mahavir Singh Khanna, New Delhi (IN); Rajesh Kumar Thaper, Jammu (IN); Mohan Prasad, Gurgaon (IN); Sudershan Kumar Arora, Gurgaon (IN)

(73) Assignee: RANBAXY LABORATORIES LIMITED, New Delhi, Delhi (IN)

(21) Appl. No.: 14/127,680

(22) PCT Filed: Jun. 20, 2012
(86) PCT No.: PCT/IB2012/053123

§ 371 Date: Aug. 20, 2014

(30) Foreign Application Priority Data
Jun. 21, 2011 (IN) 1744/DEL/2011

(51) Int. Cl. C07D 401/12

(52) U.S. Cl. CPC C07D 401/12
USPC 546/273.7

(57) ABSTRACT

The present invention relates to a process for the preparation of dexlansoprazole.xH₂O, wherein x is about 0.0 to about 0.1, using dexlansoprazole.xH₂O, wherein x is about 2.6 to about 50.

### FIELD OF THE INVENTION

The present invention relates to a process for the preparation of dexlansoprazole.xH₂O, wherein x is about 0.0 to about 0.1.

### BACKGROUND OF THE INVENTION

Dexlansoprazole is chemically 2-[(R)-{[3-methyl-4-(2,2,2-trifluoroethoxy)pyridin-2-yl]methyl}sulfinyl]-1H-benzimidazole as represented by Formula I.

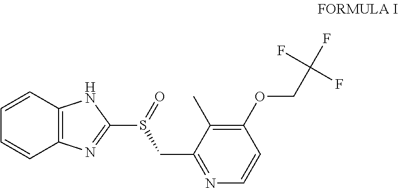

U.S. Pat. Nos. 6,462,058, and 7,285,668 and US Patent Application No. 2007/0004779 describe processes for preparing crystalline forms of dexlansoprazole and its hydrates. PCT Publication No. WO 2009/117489 describes a process for the preparation of amorphous dexlansoprazole.

U.S. Pat. No. 7,271,182 describes sodium salt, magnesium salt, lithium salt, potassium salt, calcium salt, or barium salt of dexlansoprazole and their preparation method.

U.S. Pat. No. 7,169,799 describes processes for preparing crystal of (R)-2-[[[3-methyl-4-(2,2,2-trifluoroethoxy)-2-pyridyl]methyl]sulfinyl]benzimidazole.n′H₂O (wherein n′ is about 0 to about 0.1) or a salt thereof by crystallization from an organic solvent solution or suspension in which (R)-2-R[3-methyl-4-(2,2,2-trifluoroethoxy)-2-pyridyl]methyl]sulfinyl]benzimidazole.nH₂O (wherein n is about 0.1 to about 1.0) or a salt thereof has been dissolved or suspended.

### SUMMARY OF THE INVENTION

The present inventors have found that the dexlansoprazole.xH₂O, wherein x is about 2.6 to about 50 can be converted into dexlansoprazole.xH₂O, wherein x is about 0.0 to about 0.1. By employing the present invention, dexlansoprazole.xH₂O, wherein x is about 0.0 to about 0.1, can also be obtained as chirally and chemically pure material in a consistent manner. Thus, the present invention provides a simple, efficient and industrially preferable process for the preparation of dexlansoprazole.xH₂O, wherein x is about 0.0 to about 0.1.

### DETAILED DESCRIPTION OF THE INVENTION

One aspect of the present invention provides a process for the preparation of dexlansoprazole.xH₂O, wherein x is about 2.6 to about 50, which comprises:

a) treating a salt of dexlansoprazole with an agent capable of liberating dexlansoprazole as a free base in the presence of a solvent;

b) treating the dexlansoprazole obtained in step a) with water and a solvent selected from the group consisting of halogenated hydrocarbon, ketone, C₁₋₃ alkanol, ether and a mixture thereof; and

c) isolating dexlansoprazole.xH₂O, wherein x is about 2.6 to about 50 from the mixture thereof.

The salt of dexlansoprazole used as a starting material may be in any solid form and prepared according to the methods described in U.S. Pat. No. 7,271,182. The salt may be, for example, sodium salt of dexlansoprazole. The salt of dexlansoprazole is treated with an agent capable of liberating dexlansoprazole as a free base in the presence of a solvent. The agent capable of liberating dexlansoprazole as a free base may be an acid, for example, hydrochloric acid, amine salt, for example, ammonium halide, or a hydrogen sulfate, for example, sodium or potassium hydrogen sulfate. The solvent used in step a) or step b) may be water, water-miscible solvent, for example, acetone, C₁₋₃ alkanol, dioxane, tetrahydrofuran, dimethylformamide, acetonitrile, dimethylsulfoxide or water immiscible solvent, for example, halogenated hydrocarbon, dichloromethane, or a mixture thereof.

The reaction mixture obtained in step a) or step b) may preferably be treated with water, dichloromethane, acetone, or a mixture thereof. The liberation of dexlansoprazole as a free base may be effected by stirring the reaction mixture. The reaction mixture may be treated with ammonia, for example, aqueous ammonia or an alkyl amine, for example, diisopropylethylamine in the presence of a ketone solvent, for example, acetone. The dexlansoprazole.xH₂O, wherein x is about 2.6 to about 50 obtained as a free base may optionally be isolated by solvent removal.

Another aspect of the present invention provides a process for the preparation of dexlansoprazole.xH₂O, wherein x is about 0.0 to about 0.1, which comprises:

a) treating a salt of dexlansoprazole with an agent capable of liberating dexlansoprazole as a free base in the presence of a solvent;

b) treating the dexlansoprazole obtained in step a) with water and a solvent selected from the group consisting of C₄₋₈ hydrocarbon, halogenated hydrocarbon, ketone, C₁₋₃ alkanol, ether and a mixture thereof;

c) isolating dexlansoprazole.xH₂O, wherein x is about 2.6 to about 50 from the mixture thereof; and

d) isolating dexlansoprazole.xH₂O, wherein x is about 0.0 to about 0.1 by crystallization from solvent, solution or suspensions in which dexlansoprazole.xH₂O, wherein x is about 2.6 to about 50, has been dissolved or suspended.

The salt of dexlansoprazole used as a starting material may be in any solid form and prepared according to the methods described in U.S. Pat. No. 7,271,182. The salt may be, for example, sodium salt of dexlansoprazole. The salt of dexlansoprazole is treated with an agent capable of liberating dexlansoprazole as a free base in the presence of a solvent. The agent capable of liberating dexlansoprazole as a free base may be an acid, for example, hydrochloric acid, amine salt, for example, ammonium halide, or a hydrogen sulfate, for example, sodium or potassium hydrogen sulfate. The solvent used in step a) or step b) may be water, water miscible solvent, for example, acetone, C₁₋₃ alkanol, dioxane, tetrahydrofuran, dimethylformamide, acetonitrile, dimethylsulfoxide or water immiscible solvent, for example, halogenated hydrocarbon, dichloromethane, or a mixture thereof.

The reaction mixture obtained in step a) or step b) may preferably be treated with water, dichloromethane, acetone or a mixture thereof. The liberation of dexlansoprazole as a free base may be effected by stirring the reaction mixture. The reaction mixture may be treated with ammonia, for example, aqueous ammonia or an alkyl amine, for example, diisopropylethylamine in the presence of a ketone solvent, for example, acetone. The dexlansoprazole.xH₂O, wherein x is about 2.6 to about 50 obtained as a free base may optionally be isolated by solvent removal.

The dexlansoprazole.xH₂O, wherein x is about 2.6 to about 50 isolated in step c) may be treated with a solvent. The solvent used in step d) may be selected from the group consisting of water, C₁₋₇ alkanol, halogenated hydrocarbon, ketone, aliphatic hydrocarbon, cyclic aliphatic hydrocarbon, ether and a mixture thereof. The solvent may be, for example, n-butanol, tertiary-butanol, cyclohexane, dichloromethane, acetone, heptane, methanol, methyl t-butyl ether, diisopropyl ether or a mixture thereof. The treatment with the solvent may be carried out at a temperature of about −30° C. to about 60° C., for example, about 15° C. to about 45° C. The dexlansoprazole.xH₂O, wherein x is about 0.0 to about 0.1, may be isolated by filtration, distillation, decantation, vacuum drying, evaporation, or a combination thereof.

Another aspect of the present invention provides a process for the preparation of dexlansoprazole.xH₂O, wherein x is about 0.0 to about 0.1, which comprises:

a) treating dexlansoprazole.xH₂O, wherein x is about 2.6 to about 50, with a solvent selected from the group consisting of water, C₁₋₇ alkanol, aliphatic hydrocarbon, cyclic aliphatic hydrocarbon, halogenated hydrocarbon, ketone, ether and a mixture thereof; and

b) isolating dexlansoprazole.xH₂O, wherein x is about 0.0 to about 0.1 from the mixture thereof.

The dexlansoprazole.xH₂O, wherein x is about 2.6 to about 50, is treated with a solvent selected from the group consisting of water, C₁₋₇ alkanol, halogenated hydrocarbon, ketone, aliphatic hydrocarbon, cyclic aliphatic hydrocarbon, ketone, ether, and a mixture thereof. The solvent may be, for example, n-butanol, tertiary-butanol, cyclohexane, dichloromethane, acetone, heptane, methanol, methyl t-butyl ether, diisopropyl ether, or a mixture thereof. The treatment with the solvent may be carried out at a temperature of about −30° C. to about 60° C., for example, about 15° C. to about 45° C. The dexlansoprazole.xH₂O, wherein x is about 0.0 to about 0.1 may be isolated by filtration, distillation, decantation, vacuum drying, evaporation, or a combination thereof.

Another aspect of present invention provides dexlansoprazole.xH₂O, wherein x is about 2.6 to about 50.

While the present invention has been described in terms of its specific embodiments, certain modifications and equivalents will be apparent to those skilled in the art and are intended to be included within the scope of the present invention.

### EXAMPLES

### Example 1

### Preparation of Dexlansoprazole.xH₂O, Wherein x is About 27

Dexlansoprazole sodium (300 g) was dissolved in de-ionized water (15 L) at 26° C. to 30° C. and the pH of the reaction mixture was adjusted to 12.4 to 12.6 using sodium hydroxide (100 g). The reaction mixture was heated to 45° C. to 50° C., stirred for 30 minutes and filtered through Celite-bed and filtrate was cooled to 35° C. to 38° C. The filtrate was extracted with dichloromethane (2×1200 mL). The pH of the aqueous reaction mixture was adjusted to 7.4 to 7.8 with dropwise addition of 2N hydrochloric acid (1485 mL). The reaction mixture was filtered, washed with water (1500 mL) and added to acetone (900 mL). De-ionized water (300 mL) and aqueous ammonia (22.8 mL) were added to this reaction mixture and heated to 35° C. to 38° C. De-ionized water (4.8 L) was added dropwise over a period of 45 minutes to 60 minutes. The reaction mixture was stirred for 3 hours to 4 hours at 35° C. to 38° C. and the precipitate obtained was filtered and washed with water (600 mL). The precipitate was again added to acetone (900 mL) followed by addition of de-ionized water (300 mL) and aqueous ammonia (22.8 mL). The reaction mixture was heated to 35° C. to 38° C. De-ionized water (4.8 L) was added to the reaction mixture drop-wise over a period of 45 minutes to 60 minutes. The reaction mixture was stirred for 3 hours to 4 hours at 35° C. to 38° C. and the precipitate obtained was filtered and washed with water (600 mL) to obtain the title product.

### Yield: 402 g

### Moisture: 57.0%

### Example 2

### Preparation of Dexlansoprazole.xH₂O, Wherein x is About 0.0 to about 0.1

Dexlansoprazole (402 g) prepared according to Example 1 was dissolved in dichloromethane (1500 mL) and washed with 5% aqueous sodium chloride solution (1800 mL). Layers obtained were separated and washed with de-ionized water (1800 mL).

Organic layer was separated and filtered through Celite bed followed by washing with dichloromethane (300 mL). Diisopropylethylamine (0.3 g) was added to the combined dichloromethane layer (1800 mL). n-Butanol (360 mL) and activated carbon were added to the reaction mixture and stirred for 30 minutes. The reaction mixture was filtered through celite and a bed of molecular sieve (120 g) to get moisture of organic layer not more than 0.25% w/w. Solvents were recovered completely under vacuum at less than 35° C. to get the residue. Cyclohexane (2×360 mL) was added to the residue. The cyclohexane was recovered completely from the reaction mixture under vacuum at less than 35° C. to get the residue. Cyclohexane (4300 mL) was added to the residue dropwise and the solution was stirred for 4 hours at 25° C. to 30° C. The reaction mixture was filtered. Cyclohexane (600 mL) was added to the solid material and the reaction mixture was stirred for 30 minutes at 25° C. to 30° C., filtered under nitrogen atmosphere and dried under vacuum at 35° C. to 38° C. for 10 hours to 12 hours to obtain the title compound.

### Yield: 138 g

Moisture: 0.14%

We claim:

1. A process for the preparation of dexlansoprazole.xH₂O, wherein x is about 2.6 to about 50, which comprises:
a) treating a salt of dexlansoprazole with an agent capable of liberating dexlansoprazole as a free base in the presence of a solvent;
b) treating the dexlansoprazole obtained in step a) with water and a solvent selected from the group consisting of halogenated hydrocarbon, ketone, C₁₋₃ alkanol, ether and a mixture thereof; and
c) isolating dexlansoprazole.xH₂O, wherein x is about 2.6 to about 50 from the mixture thereof.

2. A process for the preparation of dexlansoprazole.xH₂O, wherein x is about 0.0 to about 0.1, which comprises:
a) treating a salt of dexlansoprazole with an agent capable of liberating dexlansoprazole as a free base in the presence of a solvent;
b) treating the dexlansoprazole obtained in step a) with water and a solvent selected from the group consisting of C₄₋₈ hydrocarbon, halogenated hydrocarbon, ketone, C₁₋₃ alkanol, ether and a mixture thereof;
c) isolating dexlansoprazole.xH₂O, wherein x is about 2.6 to about 50 from the mixture thereof; and
d) isolating dexlansoprazole.xH₂O, wherein x is about 0.0 to about 0.1 by crystallization from solvent, solution or suspensions in which dexlansoprazole.xH₂O, wherein x is about 2.6 to about 50, has been dissolved or suspended.

3. A process according to claim 1 or claim 2, wherein the salt of dexlansoprazole is sodium salt.

4. A process according to claim 1 or claim 2, wherein the agent capable of liberating dexlansoprazole as a free base is an acid, amine salt or hydrogen sulfate.

5. A process according to claim 4, wherein the agent capable of liberating dexlansoprazole is hydrochloric acid.

6. A process according to claim 1 or claim 2, wherein the solvent used in step a) is water, halogenated hydrocarbon, or a mixture thereof.

7. A process according to claim 6, wherein the halogenated hydrocarbon is dichloromethane.

8. A process according to claim 1 or claim 2, wherein the halogenated hydrocarbon used in step b) is dichloromethane.

9. A process according to claim 1 or claim 2, wherein the ketone used in step b) is acetone.

10. A process according to claim 1 or claim 2, wherein the ether used in step b) is tetrahydrofuran.

11. A process according to claim 2, wherein the solvent used in step d) is selected from the group consisting of water, C₁₋₇ alkanol, halogenated hydrocarbon, ketone, aliphatic hydrocarbon, cyclic aliphatic hydrocarbon, ether, and a mixture thereof.

12. A process according to claim 11, wherein the solvent used in step d) is n-butanol, tertiary-butanol, cyclohexane, dichloromethane, acetone, heptane, methanol, methyl t-butyl ether, diisopropyl ether, or a mixture thereof.

13. A process for the preparation of dexlansoprazole.xH₂O, wherein x is about 0.0 to about 0.1, which comprises:
a) treating dexlansoprazole.xH₂O, wherein x is about 2.6 to about 50, with solvent selected from the group consisting of C₁₋₇ alkanol, aliphatic hydrocarbon, cyclic aliphatic hydrocarbon, halogenated hydrocarbon, ketone, ether, and a mixture thereof; and
b) isolating dexlansoprazole.xH₂O, wherein x is about 0.0 to about 0.1 from the mixture thereof.

14. A process according to claim 13, wherein the solvent is n-butanol, tertiary-butanol, cyclohexane, dichloromethane, acetone, heptane, methanol, methyl t-butyl ether, diisopropyl ether, or a mixture thereof.

15. Dexlansoprazole.xH₂O, wherein x is about 2.6 to about 50.
